(19) United States
(12) Patent Application Publication
(10) Pub. No.: US 2026/0290583 A1
(43) Pub. Date: Sep. 24, 2026

(54) METHOD FOR ANALYZING GENETIC RELATIONSHIPS, AND SYSTEM THEREFOR

(71) Applicant: CURIGIN CO.,LTD., Seoul (KR)

(72) Inventors: Jin Woo CHOI, Seoul (KR); Ye Ji SUN, Seoul (KR)

(73) Assignee: CURIGIN CO.,LTD., Seoul (KR)

(21) Appl. No.: 19/136,470

(22) PCT Filed: Dec. 8, 2023
(86) PCT No.: PCT/KR2023/020222

§ 371 Date: Jun. 3, 2026

(30) Foreign Application Priority Data
Dec. 8, 2022 (KR) 10-2022-0170552
Dec. 8, 2022 (KR) 10-2022-0170556
Dec. 8, 2022 (KR) 10-2022-0170620

(51) Int. Cl. G16H 50/20; G16B 20/20; G16B 25/10; G16B 50/30; G16H 20/10; G16H 50/70

(52) U.S. Cl. CPC G16H 50/20; G16B 20/20; G16B 25/10; G16B 50/30; G16H 20/10; G16H 50/70

(57) ABSTRACT

Provided are a method for analyzing genetic relationships and a system therefor. A method for analyzing genetic relationships, according to some embodiments, comprises the steps of: obtaining gene expression data and prognostic data of patients associated with a specific disease or condition; identifying disease or condition-specific genes associated with the specific disease or condition on the basis of the gene expression data and prognostic data; analyzing correlations between the disease or condition-specific genes to identify candidate gene pairs in which positive or inverse correlations between the disease or condition-specific genes can be established; analyzing hierarchical relationship between the two genes constituting each of the candidate gene pairs; and building a gene network associated with the specific disease or condition on the basis of the results of the analysis.

### TECHNICAL FIELD

The disclosed embodiments relate to analyzing relationships between genes associated with a specific disease.

### BACKGROUND ART

Cancer (tumor), especially lung cancer, is a disease with a high mortality rate, and attempts to discover biomarkers through prognostic analysis are ongoing, but there are still difficulties in discovering clinical gene targets. Genome analysis can be used to discover these targets, and many researchers are trying to identify relationships between genes associated with cancer (tumors) through genome analysis based on massive public data.

With advances in technologies such as RNA sequencing, microarrays, and ChIP-Seq, researchers can understand biological processes within cells by measuring gene expression. In addition, researchers identify and analyze genes associated with cancer (tumors) through pathways, ontology analysis, or multi-omics-based approaches to identify intergenic networks.

However, although information about gene expression patterns is abundant, information indicating interrelationships or causal relationships between genes is limited. Therefore, a new perspective algorithm is required that can accurately investigate complex relationships between genes.

### DISCLOSURE

### Technical Problem

An object to be solved through some embodiments of the present disclosure is to provide a method and a system thereof capable of accurately analyzing relationships between genes associated with a specific disease (e.g., cancer) or condition.

Another object to be solved through some embodiments of the present disclosure is to provide a method and a system thereof capable of accurately building a network of genes associated with a specific disease (e.g., cancer) or condition.

Yet another object to be solved through some embodiments of the present disclosure is to provide a method and a system thereof capable of accurately identifying a biomarker and/or therapeutic target gene for a specific disease (e.g., cancer) or condition.

The objects of the present disclosure are not restricted to the aforementioned objects, and other objects of the present disclosure, which are not mentioned above, will be clearly understood by those skilled in the art from the description below.

### Technical Solution

In order to solve the above-described object, according to an aspect of the present disclosure, there is provided a method for analyzing genetic relationships, the method performed by at least one computing device, which may include the steps of: obtaining gene expression data and prognostic data of patients associated with a specific disease; identifying disease-specific genes associated with the specific disease on the basis of the gene expression data and the prognostic data; analyzing correlations between the disease-specific genes to identify candidate gene pairs in which positive or inverse correlations between the disease-specific genes are established; analyzing a hierarchical relationship between the two genes constituting each of the candidate gene pairs; and building a gene network associated with the specific disease on the basis of the results of the analysis.

In some embodiments, the identifying of the disease-specific genes may include: analyzing the prognosis of a first patient group and a second patient group on the basis of the gene expression data and the prognostic data, in which the first patient group is a patient group in which the expression level of a specific gene is higher than that of the second patient group; and identifying the specific gene as a disease-specific gene on the basis of the analysis results of the prognosis.

In some embodiments, the analyzing of the hierarchical relationship between the two genes may include: identifying the first gene as a upstream gene and identifying the second gene as a downstream gene when the number of candidate gene pairs to which the first gene belongs is greater than or equal to the number of candidate gene pairs to which the second gene belongs.

In some embodiments, the analyzing of the hierarchical relationship between the two genes may include: dividing the two genes into the upstream gene and the downstream gene on the basis of the number of differentially expressed genes (DEGs) associated with each of the two genes.

In some embodiments, the analyzing of the hierarchical relationship between the two genes may include: dividing the two genes into the upstream gene and the downstream gene by analyzing a temporal relationship between the expressions of the two genes according to a specific drug on the basis of drug duration data.

In some embodiments, the analyzing of the hierarchical relationship between the two genes may include: dividing the two genes into the upstream gene and the downstream gene by analyzing a relationship between changes in the expression levels of the two genes according to a specific shRNA on the basis of gene expression data on short hairpin RNA (shRNA) knockdown.

In some embodiments, the building of the gene network associated with the specific disease may include: generating a first node and a second node corresponding to each of a first gene identified as a upstream gene and a second gene identified as a downstream gene of the two genes as a result of the analysis; and building the gene network by connecting the first node and the second node with a directed edge, in which the directed edge is an edge directed from the first node to the second node.

In some embodiments, according to another aspect of the present disclosure for solving the technical object described above, there is provided a genetic relationship analysis system including: one or more processors; and a memory storing a computer program executed by the one or more processors, in which the computer program may include instructions for the operations of: obtaining gene expression data and prognostic data of patients associated with a specific disease; identifying disease-specific genes associated with the specific disease on the basis of the gene expression data and the prognostic data; analyzing correlations between the disease-specific genes to identify candidate gene pairs in which positive or inverse correlations between the disease-specific genes are established; analyzing hierarchical relationship between the two genes constituting each of the candidate gene pairs; and building a gene network associated with the specific disease on the basis of the results of the analysis.

In order to solve the object described above, according to yet another aspect of the present disclosure, there is provided a non-transitory computer-readable recording medium recorded with a computer program for analyzing genetic relationships, in which the computer program may include instructions for the operations of: obtaining gene expression data and prognostic data of patients associated with a specific disease; identifying disease-specific genes associated with the specific disease on the basis of the gene expression data and the prognostic data; analyzing correlations between the disease-specific genes to identify candidate gene pairs in which positive or inverse correlations between the disease-specific genes are established; analyzing hierarchical relationship between the two genes constituting each of the candidate gene pairs; and building a gene network associated with the specific disease on the basis of the results of the analysis.

### Advantageous Effects

According to the disclosed embodiments, it is possible to accurately identify specific genes (e.g., cancer-specific genes) associated with a specific disease or condition by analyzing gene expression data and prognostic data of patients associated with a specific disease (e.g., cancer) or condition. The specific genes identified above can be used as biomarkers and/or therapeutic target genes for a specific disease or condition. For example, the identified disease or condition-specific genes (cancer-specific genes) can be used as biomarkers and/or therapeutic target genes for a specific disease or condition.

In addition, it is possible to accurately identify specific genes associated with a specific disease or condition by comparing and analyzing prognosis between patient groups with high and low expression of specific genes.

In addition, it is possible to accurately identify candidate gene pairs associated with each other by performing correlation analysis on pairs of identified specific genes.

In addition, it is possible to accurately analyze (investigate) the hierarchical relationship between two genes constituting a candidate gene pair on the basis of the number of differentially expressed genes (DEGs).

In addition, it is possible to accurately analyze (investigate) the hierarchical relationship between two genes constituting a candidate gene pair by analyzing a temporal relationship between the expressions of genes that are shown according to drug administration. For example, it is possible to first identify the gene whose expression changes as an upstream gene, and then identify a following gene as a downstream gene.

In addition, it is possible to accurately analyze (investigate) the hierarchical relationship between two genes constituting a candidate gene pair by analyzing a relationship between changes in the expression levels of genes that occur according to short hairpin RNA (shRNA) knockdown.

In addition, it is possible to accurately identify genes corresponding to upstream (source) or downstream (target) without using existing algorithms, well-known pathways, or multi-omics.

In addition, it is possible to accurately build a gene network associated with a specific disease or condition on the basis of the results of analyzing a hierarchical relationship between candidate gene pairs. Such a gene network can be useful for discovering biomarkers and/or therapeutic target genes for a specific disease (e.g., cancer) or condition, and can also be useful for understanding complex expression relationships between genes associated with a specific disease (e.g., cancer) or condition and a mechanisms of action of drugs (e.g., therapeutic agents such as anticancer drugs) associated with a specific disease (e.g., cancer). Furthermore, specific characteristics of cancer may also be described through a gene network.

In addition, as the hierarchical relationship between genes is investigated, biomarkers that can be applied in actual clinical practice (e.g., previously unknown new biomarkers) can be easily discovered. In addition, top-level gene expression regulators can be easily discovered, which may provide significant help in treating a patient's disease. In addition, a new relationship between genes can also be discovered. Furthermore, it is possible to more accurately predict therapeutic targets associated with a specific disease by building a more practical and accurate gene network than existing approaches.

The effects according to embodiments of the present disclosure are not limited to the aforementioned effects, and other effects, which are not mentioned above, will be apparently understood to those skilled in the art from the following disclosure.

### DESCRIPTION OF DRAWINGS

FIG. 1 is an exemplary diagram for describing an operation of a system for analyzing genetic relationships at a system level according to some embodiments of the present disclosure.

FIG. 2 is an exemplary flowchart illustrating a method for analyzing genetic relationships according to some embodiments of the present disclosure.

FIG. 3 is an exemplary diagram for describing a method for dividing patients associated with a specific cancer into a high-expression patient group and a low-expression patient group on the basis of gene expression levels according to some embodiments of the present disclosure.

FIGS. 4 and 5 are exemplary diagrams for describing a method for analyzing survival rates between high-expression patient groups and low-expression patient groups for a specific gene according to some embodiments of the present disclosure.

FIG. 6 is an exemplary diagram for describing a method for analyzing correlations between cancer-specific genes according to some embodiments of the present disclosure.

FIG. 7 illustrates candidate gene pairs having positive correlations.

FIGS. 8 and 9 are exemplary diagrams for describing a method for analyzing a hierarchical relationship between genes according to some embodiments of the present disclosure.

FIGS. 10 and 11 are exemplary diagrams for describing a method for analyzing a hierarchical relationship between genes according to some other embodiments of the present disclosure.

FIG. 12 illustrates a gene network built through the method for analyzing the hierarchical relationship between genes described with reference to FIGS. 10 and 11.

FIGS. 13 and 14 are exemplary diagrams for describing a method for analyzing a hierarchical relationship between genes according to some other embodiments of the present disclosure.

FIG. 15 illustrates a gene network built through the method for analyzing the hierarchical relationship between genes described with reference to FIGS. 13 and 14.

FIG. 16 schematically illustrates a genetic relationship analysis process performed in Experimental Examples conducted by the present inventors.

FIG. 17 illustrates an exemplary computing device capable of implementing a system for analyzing genetic relationships according to some embodiments of the present disclosure.

### MODES

Hereinafter, embodiments of the present disclosure will be described in detail with reference to the accompanying drawings. The same reference numbers or symbols presented in each drawing represent parts or components that perform substantially the same functions.

Terms including ordinary numbers, such as first and second, are used for describing various components, but the components are not limited by these terms. The terms are used only to discriminate one component from the other component. For example, without departing from the scope of the present disclosure, a first component may be named as a second component, and similarly, a second component may also be named as a first component. A term ‘and/or’ includes a combination of a plurality of associated items or any item of the plurality of associated items.

The terms used herein are used for describing embodiments, and are not intended to limit and/or restrict the present disclosure. A singular expression includes a plural expression unless the context indicates otherwise. In the present disclosure, it should be understood that the term “including” or “having” indicates that features, numbers, steps, operations, components, parts or combinations thereof described in the specification are present, but does not exclude the possibility of presence or addition of one or more other features, numbers, steps, operations, components, parts or combinations thereof, in advance.

In the present disclosure, when a part is connected to the other part, this includes not only cases where the parts are directly connected, but also cases where the parts are electrically connected with another element interposed therebetween. Further, unless explicitly described to the contrary, when any part includes any component, it will be understood to further include another component rather than excluding another component.

In addition, the terms including “part”, “module”, “device”, and the like disclosed in the specification mean a unit that processes at least one function or operation, which may be implemented by hardware, software or a combination of hardware and software.

Hereinafter, various embodiments of the present disclosure will be described in detail with reference to the accompanying drawings.

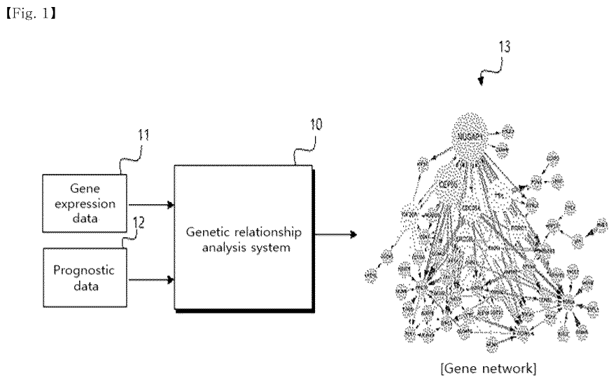

FIG. 1 is an exemplary diagram for describing an operation of a genetic relationship analysis system 10 at a system level according to disclosed embodiments.

As illustrated in FIG. 1, the genetic relationship analysis system 10 is a computing device/system equipped with a function capable of analyzing a relationship between genes associated with a specific disease. For example, the genetic relationship analysis system 10 may analyze gene expression data 11 and prognostic data 12 of patients associated with a specific disease (e.g., cancer) to identify genes associated with a specific disease (e.g., cancer-specific genes) and analyze relationships between the genes. In addition, the genetic relationship analysis system 10 may build a gene network 13 associated with a specific disease (e.g., cancer) on the basis of the analysis results. Such a gene network 13 may be useful for discovering biomarkers and/or therapeutic target genes for a specific disease (e.g., cancer), and may also be useful for understanding complex expression relationships between genes associated with a specific disease (e.g., cancer) and a mechanisms of action of drugs (e.g., therapeutic agents such as anticancer drugs) associated with a specific disease (e.g., cancer).

Hereinafter, for the convenience of understanding, it will be described assuming that the specific disease is ‘cancer (tumor)’. In addition, for convenience of explanation, the genetic relationship analysis system 10 is abbreviated as the ‘system’.

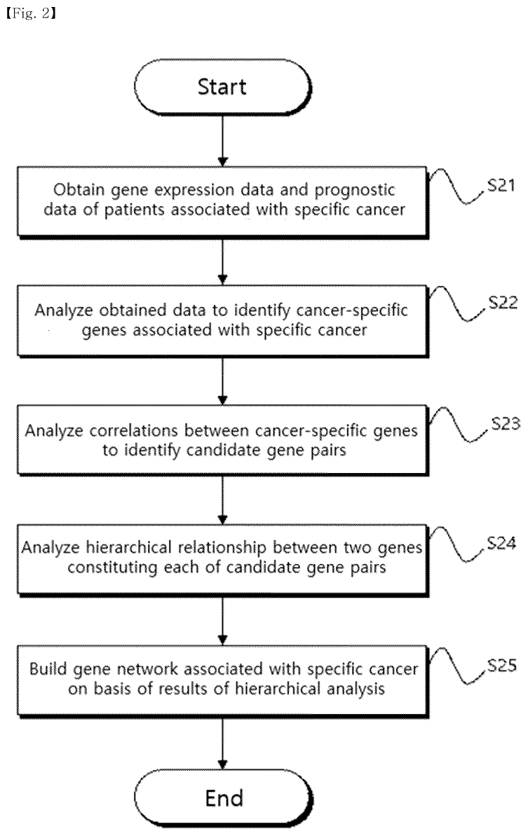

A specific method for the system 10 to identify genes associated with a specific disease and analyze relationships between the genes will be described in detail with reference to the drawings below FIG. 2.

The aforementioned system 10 may be implemented with at least one computing device. For example, the system 10 may also be implemented on one computing device, and a first function of the system 10 may be implemented on a first computing device and a second function may be implemented on a second computing device. Alternatively, a specific function of the system 10 may also be implemented in a plurality of computing devices.

The computing device may include any device having a computing (processing) function, and an example of such a device will be described with reference to FIG. 17. Since the computing device is one assembly in which a plurality of components (e.g., processor, memory, etc.) interact with each other, the computing device may also be referred to as a ‘computing system’ in some cases. Of course, the term computing system may also encompass a concept of an assembly in which a plurality of computing devices interacts with each other.

So far, the operation of the system 10 according to some embodiments of the present disclosure has been schematically described with reference to FIG. 1. Hereinafter, various methods that may be performed in the above-described system 10 will be described in detail with reference to the drawings below FIG. 2.

Hereinafter, for providing the convenience of understanding, the description will be followed assuming that all steps/operations of methods to be described below are performed in the above-described system 10. Accordingly, if the subject of a specific step (operation) is omitted, it should be understood that the methods are performed by the system 10. Of course, in an actual environment, some steps/operations of the methods to be described below may also be performed on other computing devices.

FIG. 2 is an exemplary flowchart illustrating a method for analyzing genetic relationships according to some embodiments of the present disclosure. However, it is only an exemplary embodiment for achieving the object of the present disclosure, and of course, some steps may be added or deleted as necessary.

According to the disclosed embodiments, a method for building a gene network by analyzing a hierarchical relationship between genes may be provided. To analyze the hierarchical relationship between genes and build a gene network, steps S21 to S25 illustrated in FIG. 2 may be performed.

In step S21, gene expression data and prognostic data of patients associated with a specific cancer are obtained. The prognostic data may be, for example, data on the survival rates of patients, but the scope of the present disclosure is not limited thereto.

For reference, the Cancer Genome Atlas (TCGA) project provides gene expression data and prognostic data associated with various cancer types through cBioPortal, etc.

In addition, various high-quality data may be collected and processed and applied to various embodiments of the present disclosure. For example, datasets provided by TCGA and CMAP (connectivity map) LINCS (library of integrated network-based cellular signatures) 2020 database and GSE (genomic spatial event) 106127 database may be used in various embodiments of the present disclosure.

In step S22, the obtained gene expression data and prognostic data are analyzed to identify cancer-specific genes associated with a specific cancer. For example, cancer-specific genes that are adverse to survival may be identified by analyzing gene expression data and prognostic data on the survival rate of patients associated with a specific cancer. Specifically, cancer-specific genes may be identified among differentially expressed genes by comparing and analyzing the survival rates between a high-expression patient group and a low-expression patient group for a specific gene. These cancer-specific genes may also serve as biomarkers and/or therapeutic target genes for a specific cancer.

In some cases, genes that are favor to the survival of patients may also be identified.

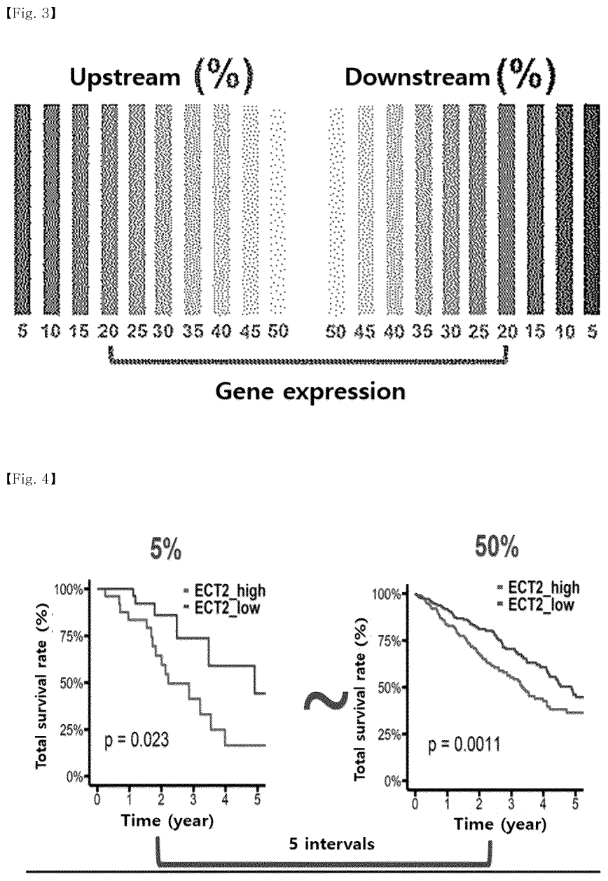

Hereinafter, the details of step S22 will be further described in more detail with reference to FIG. 3 to 5.

FIG. 3 is an exemplary diagram for describing a method for dividing patients associated with a specific cancer into a high-expression patient group and a low-expression patient group on the basis of gene expression levels according to some embodiments of the present disclosure.

As shown in FIG. 3, patients may be divided into a patient group with a high expression level of a specific gene (i.e., a high-expression patient group) and a patient group with a low expression level (i.e., a low-expression patient group) of the specific gene. For example, the patients may be divided into a high-expression patient group and a low-expression patient group at 5% intervals from 5% to 50% according to a gene expression level (however, values for dividing patient groups, the number of patient groups, etc. may be changed at any time). Specifically, among patients, according to the expression level of a specific gene, a group of patients with the top 5% and a group of patients with the bottom 5% may be divided, and a group of patients with the top 10% and a group of patients with the bottom 10% may be divided. Here, the group of patients with the top 10% may also mean, for example, patients whose gene expression levels fall in the top 5% to 10%, or may also mean all patients whose gene expression levels fall within the top 10%. In this way, patient groups of top/bottom 5%, 10%, 15%, 20%, 25%, 30%, 35%, 40%, 45%, and 50% may be divided according to an expression level of a specific gene, respectively. In addition, patient groups may be divided in the same manner for other genes and survival rates may be compared as described below.

FIG. 4 is an exemplary diagram for describing a method for analyzing survival rates between high-expression patient groups and low-expression patient groups for a specific gene (e.g., ECT2) according to some embodiments of the present disclosure.

When high-expression patient groups (e.g., top 5%, 10%, etc.) and low-expression patient groups (e.g., bottom 5%, 10%, etc.) are divided as illustrated in FIG. 3, the survival rates of the two patient groups over time may be compared, as illustrated in FIG. 4. For example, the survival rates of two patient groups over time may be compared a total of 10 times. Specifically, there may be performed a comparison of first survival rates between top 5% patient groups and bottom 5% patient groups, a comparison of second survival rates between top 5% to 10% patient groups and bottom 5% to 10% patient groups, a comparison of third survival rates between top 10% to 15% patient groups and bottom 10% to 15% patient groups, a comparison of fourth survival rates between top 15% to 20% patient groups and bottom 15% to 20% patient groups, a comparison of fifth survival rates between top 20% to 25% patient groups and bottom 20% to 25% patient groups, a comparison of sixth survival rates between top 25% to 30% patient groups and bottom 25% to 30% patient groups, a comparison of seventh survival rates between top 30% to 35% patient groups and bottom 30% to 35% patient groups, a comparison of eighth survival rates between top 35% to 40% patient groups and bottom 35% to 40% patient groups, a comparison of ninth survival rates between top 40% to 45% patient groups and bottom 40% to 45% patient groups, and a comparison of tenth survival rates between top 45% to 50% patient groups and bottom 45% to 50% patient groups. Cancer-specific genes (i.e., genes that are adverse to survival when expressed high) may be accurately identified by repeatedly performing a total of 10 survival rate comparisons for high-expression patient groups and low-expression patient groups.

Since the cancer-specific genes identified above are more likely than other genes to affect the cause of a specific cancer or the malignancy of the cancer, there is a possibility to treat the specific cancer by inhibiting the corresponding genes, and in this respect, the cancer-specific genes may be used as therapeutic target genes. In addition, the identified cancer-specific genes may serve as indicators of the prognosis of the specific cancer and thus may also be used as biomarkers for the specific cancer.

As described above, in some cases, genes that are favor to the survival when the expression is high may also be identified.

As a more specific example, the survival rates of patient groups with top X % and bottom X % gene expression levels for approximately 20,000 genes provided by TCGA may be repeatedly compared and analyzed. For example, the survival rates of patient groups with top and bottom 5%, 10%, 15%, 20%, 25%, 30%, 35%, 40%, 45%, and 50% of expression levels of specific genes are analyzed (compared), respectively, and when in the results of 10 times analyses, a reference value (e.g., approximately 7 times) or more is judged as “adverse”, the corresponding gene may be identified as a cancer-specific gene. Such an identification process may be repeated for about 20,000 genes.

When summarizing the aforementioned contents, the expression levels of specific genes are divided at 5% intervals from 5% to 50% to analyze the prognosis of high-expression patient groups and low-expression patient groups. In addition, when the survival rates of the high-expression patient groups are significantly low, specific genes may be identified (selected) as cancer-specific genes (i.e., adverse genes). In addition, such an identification process may be repeated even for other genes.

For reference, the above-described identification process may be performed using Kaplan-Meier survival analysis provided by a survival package. Specifically, through Kaplan-Meier survival analysis, interval survival rates are calculated for each interval in which an event (e.g., death) occurs, and a cumulative survival rate may be estimated using the interval survival rates. In addition, through a log-rank test, it may be determined whether there is a significant difference in cumulative survival rates between the two patient groups. Since these matters are obvious to those skilled in the art, further description will be omitted.

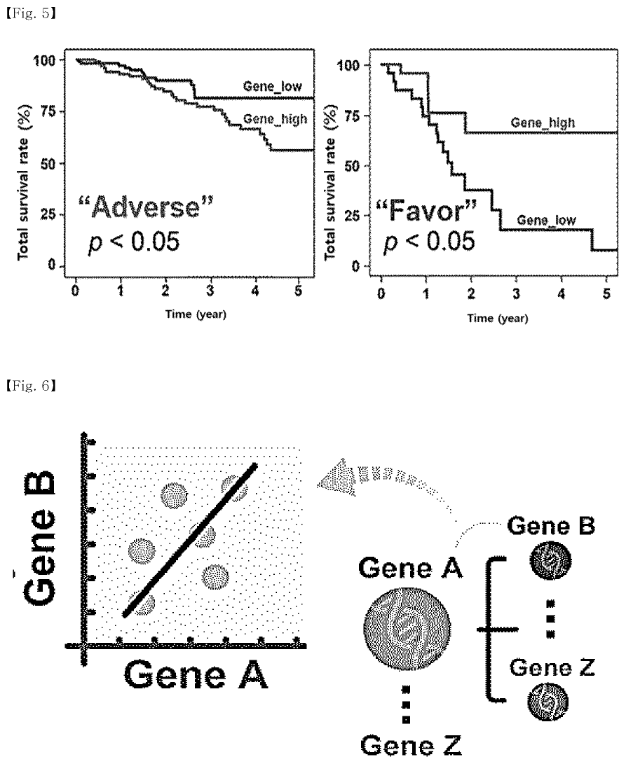

FIG. 5 illustrates criteria for dividing between genes that are adverse to survival and genes that are favor to survival when expressed at high, on the basis of the results of analyzing the survival rates of high-expression patient groups and low-expression patient groups according to some embodiments of the present disclosure.

Meanwhile, in some embodiments, an adverse score for survival rate may be calculated in a weighted sum method, and genes with an adverse score higher than or equal to a reference value may also be identified as cancer-specific genes. For example, it is assumed to compare survival rates between 10 high-expression patient groups (e.g., top 5%, etc.) and 10 low-expression patient groups (e.g., bottom 5%, etc.). In this case, an adverse score may be calculated on the basis of a difference in survival rate for each patient group. In addition, on the basis of a weighted sum of adverse scores for each patient group, the final adverse score for a specific gene may be calculated. At this time, the weight applied to the adverse score by patient group may be determined on the basis of a difference in expression level of the gene (i.e., the weight applied to the adverse score of the top 5% patient group and the bottom 5% patient group is a larger value than the weight applied to the adverse score of the top 50% patient group and the bottom 50% patient group). Accordingly, it is possible to accurately calculate the final adverse score and more accurately identify cancer-specific genes.

It will be described with reference to FIG. 2 again.

In step S23, candidate gene pairs are identified by analyzing correlations between cancer-specific genes. That is, gene pairs in which positive or inverse correlations between cancer-specific genes are established are identified as candidate gene pairs. For example, among cancer-specific genes, gene pairs with positive correlations of 0.6 or higher (i.e., correlation coefficients of 0.6 or higher) may be identified as candidate gene pairs. This is because, in gene pairs with positive correlations, if the expression of any one gene (i.e., upstream gene) is regulated, the expression of the other gene (i.e., downstream gene) is highly likely to move in the same direction. However, the scope of the present disclosure is not limited thereto.

Correlations between cancer-specific genes may be analyzed, for example, using a Pearson correlation analysis technique, but the scope of the present disclosure is not limited thereto. In addition, the Pearson correlation analysis technique may be implemented, for example, through Python and applied to step S23, but the scope of the present disclosure is not limited thereto.

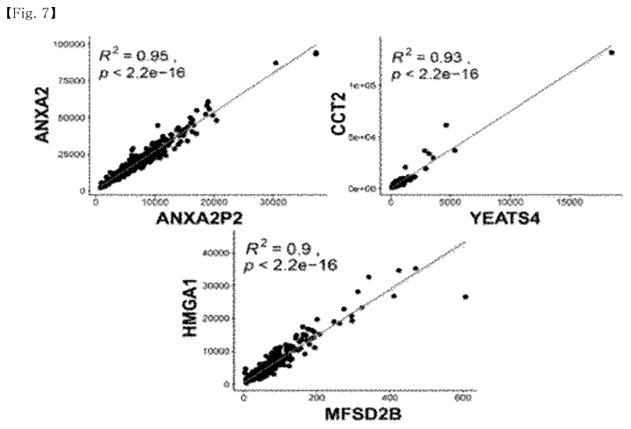

Hereinafter, step S23 will be described in more detail with reference to FIGS. 6 and 7.

FIG. 6 is an exemplary diagram for describing a method for analyzing correlations between cancer-specific genes according to some embodiments of the present disclosure.

As illustrated in FIG. 6, when the number of identified cancer-specific genes is ‘N’, correlation analysis may be performed on (N−1)+(N−2)+ . . . +1 gene pairs. For example, by confirming whether the expression values of two genes have a linear relationship, the correlations of gene pairs may be analyzed, and gene pairs with strong (high) correlations (e.g., a linear relationship of about 0.6 or higher) among cancer-specific genes may be selected (identified) as candidate gene pairs.

FIG. 7 illustrates candidate gene pairs having positive correlations.

Referring to FIG. 7, ANXA2-ANXA2P2, CCT2-YEATS4, and HMGA1-MFSD2B genes have positive correlations, and thus may be selected (identified) as candidate gene pairs. For reference, it can be confirmed that the ANXA2-ANXA2P2 genes have stronger (higher) correlations than the CCT2-YEATS4 and HMGA1-MFSD2B genes.

It will be described with reference to FIG. 2 again.

In step S24, the hierarchical relationship between the two genes constituting each of the candidate gene pairs may be analyzed. Here, the analyzing of the hierarchical relationship between the two genes may mean dividing (differentiating) upstream genes that regulate the expression of other genes (or genes that affect the expression of other genes) and downstream genes whose expression is regulated by other genes (or genes whose expression level is changed under the influence of other genes). The upstream genes and downstream genes may also be named ‘source genes’ and ‘target genes’ in some cases.

The specific method of analyzing the hierarchical relationship between two genes in step S24 may vary depending on embodiments.

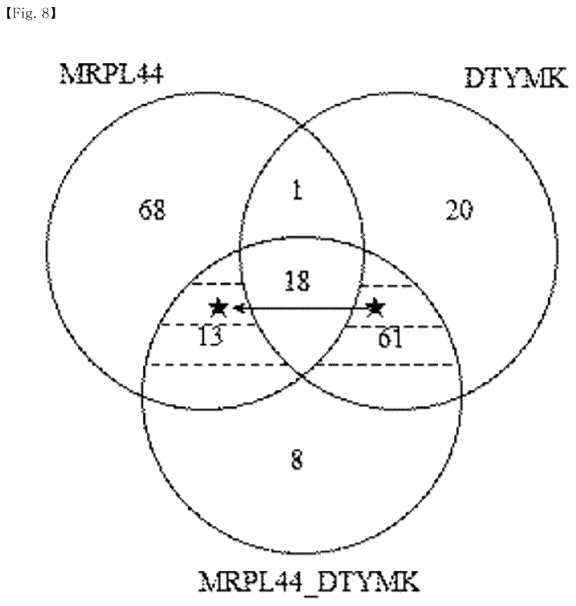

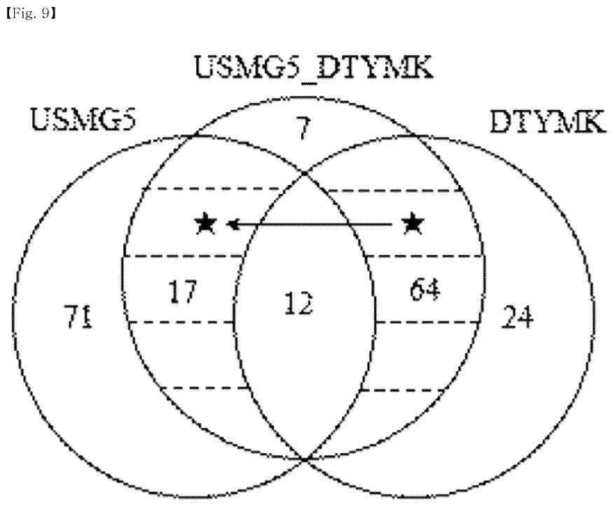

In some embodiments, the hierarchical relationship between the two genes may be analyzed on the basis of the number of differentially expressed genes associated with the two genes. This may be understood that when the number of differentially expressed genes associated with the first gene is greater than that of the second gene, the first gene is highly likely to be an upstream gene than the second gene (i.e., it is highly likely to be a control gene). These embodiments will be described below in detail with reference to FIGS. 8 and 9.

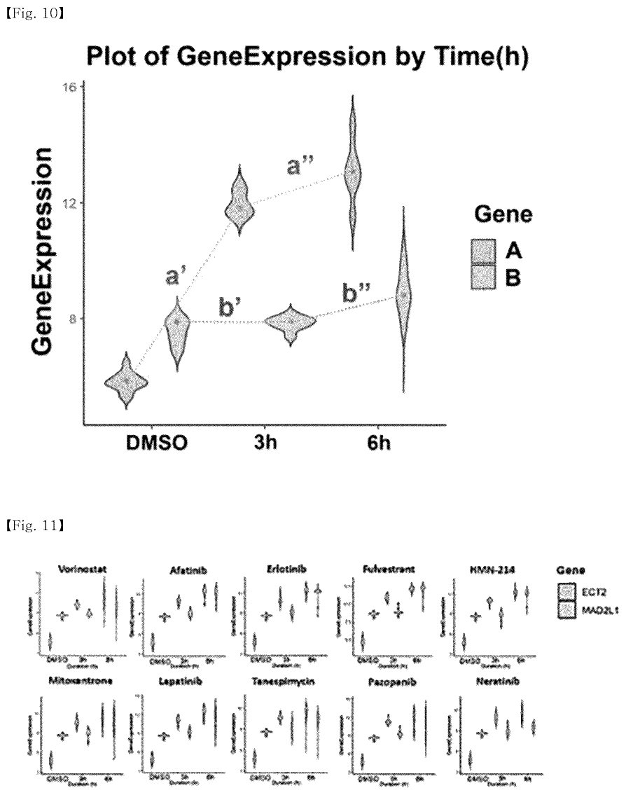

In some other embodiments, the two genes may be divided as upstream and downstream genes by analyzing a temporal relationship (difference) between the expressions of the two genes according to a specific drug. These embodiments will be described below in detail with reference to FIG. 10 to 12.

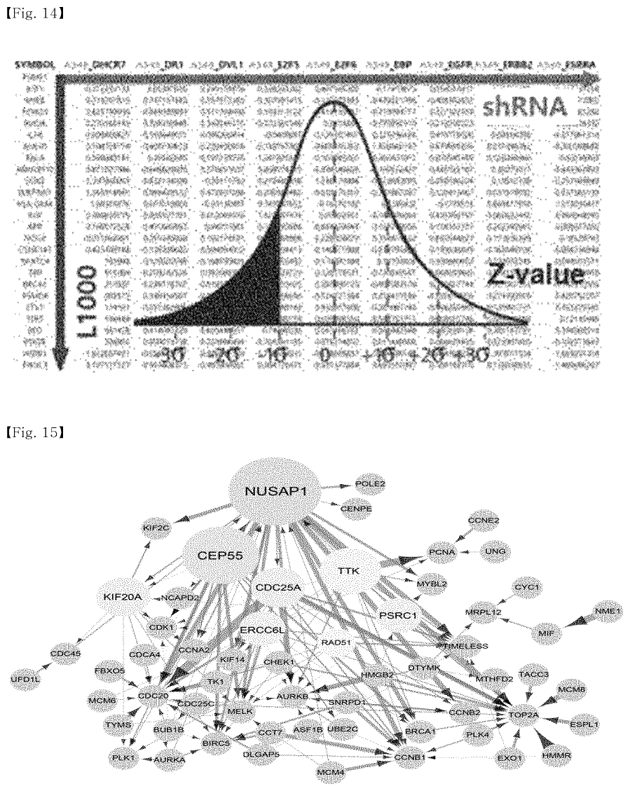

In some other embodiments, the two genes may be divided into the upstream gene and the downstream gene by analyzing a relationship (sequential expression relationship) between changes in expression levels of the two genes according to a specific shRNA on the basis of gene expression data on short hairpin RNA (shRNA) knockdown. These embodiments will be described below in detail with reference to FIG. 13 to 15.

In some other embodiments, the hierarchical relationship between the two genes may be analyzed on the basis of various combinations of the embodiments described above. For example, the hierarchical relationship between the two genes constituting the candidate gene pair may also be determined (found) by synthesizing the results of the hierarchical relationship analysis according to the first embodiment (e.g., differentially expressed gene-based analysis) and the results of the hierarchical relationship analysis according to the second embodiment (e.g., drug-based analysis).

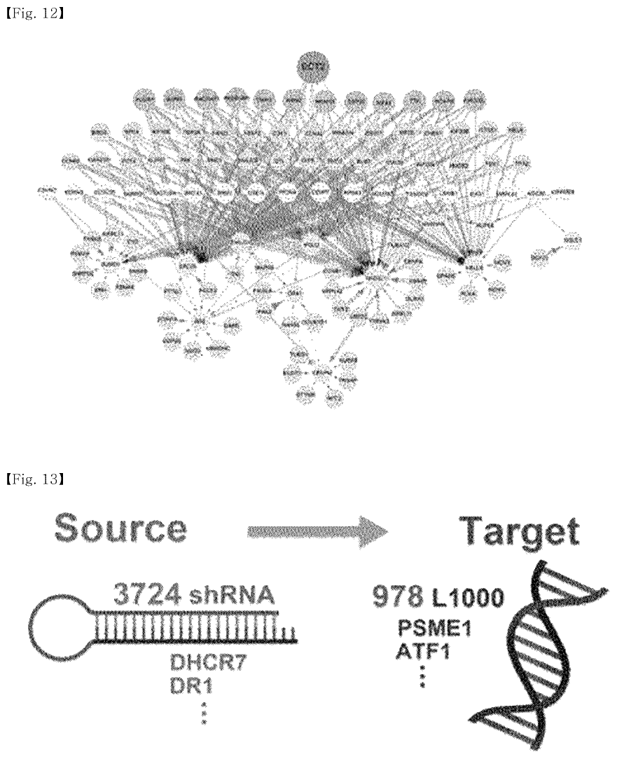

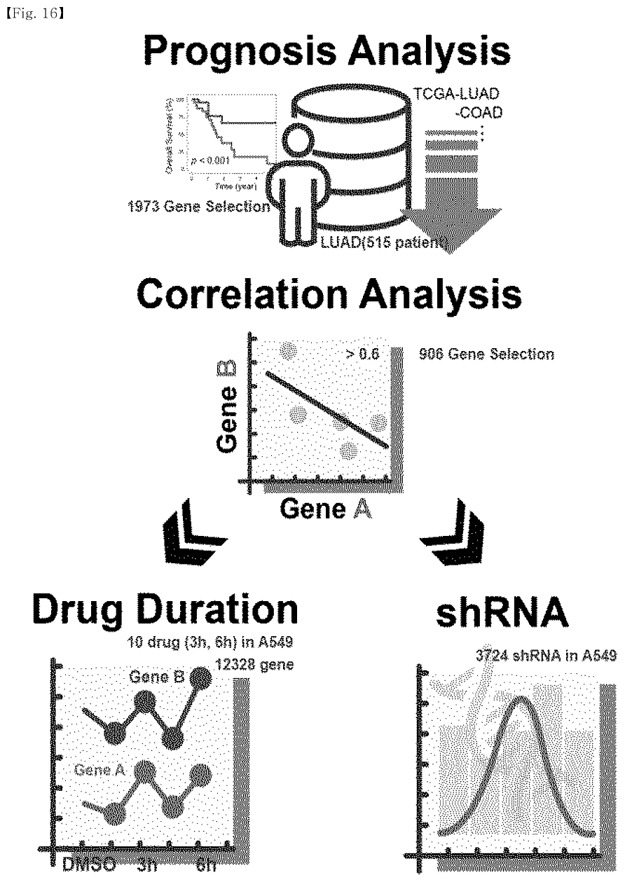

In step S25, the gene network associated with the specific cancer is established on the basis of the results of the hierarchical relationship analysis. Specifically, as the results of the hierarchical relationship analysis, upstream nodes corresponding to genes identified as the upstream genes and downstream nodes corresponding to genes identified as the downstream genes are generated and the upstream nodes and the downstream nodes are connected with directed edges (i.e., directed edges from upstream genes/nodes to downstream genes/nodes) to build a gene network associated with a specific cancer. Examples of gene networks built above will be described with reference to FIGS. 12 and 15.

In some embodiments, therapeutic target genes for a specific cancer may also be determined on the basis of a gene network. For example, among a plurality of nodes forming a gene network, nodes with an out-degree higher than or equal to a threshold are selected, and genes corresponding to the selected nodes (e.g., genes of nodes with a large number of neighboring downstream nodes and upstream genes with a large number of influencing downstream genes) may be determined as therapeutic target genes for a specific cancer (e.g., the top gene with the largest out-degree is determined as a therapeutic target gene). The genes determined above may serve as highly effective therapeutic targets. The reason is that when the expression of the corresponding gene is inhibited, the expression of a plurality of downstream genes (i.e., cancer-specific genes) may be inhibited successively. As another example, among a plurality of nodes forming a gene network, nodes with an out-degree less than or equal to a threshold are selected, and genes corresponding to the selected nodes (e.g., genes of nodes with a small number of neighboring downstream nodes and upstream genes with a small number of influencing downstream genes) may be determined as therapeutic target genes for a specific cancer (e.g., a gene with an out-degree of 0 is determined as a therapeutic target gene). The genes determined above may be significantly stable therapeutic targets (e.g., targets with very few side effects). The reason is that even if the expression of the corresponding gene is inhibited, other genes are not affected.

So far, a genetic relationship analysis method according to the disclosed embodiments has been described with reference to FIG. 2 to 7. As described above, it is possible to accurately identify specific genes (e.g., cancer-specific genes) associated with a specific disease by analyzing gene expression data and prognostic data of patients associated with a specific disease (e.g., cancer). The specific genes identified above may be used as biomarkers and/or therapeutic target genes for a specific disease. For example, the identified cancer-specific genes may be used as biomarkers and/or therapeutic target genes for specific cancer or cancer prognosis.

In addition, it is possible to accurately identify specific genes associated with a specific disease by comparing and analyzing prognosis between patient groups with high expression and low expression of specific genes.

In addition, it is possible to accurately build a gene network associated with a specific disease on the basis of the results of analyzing a hierarchical relationship between candidate gene pairs. Such a gene network may be usefully used for discovering biomarkers and/or therapeutic target genes for a specific disease (e.g., cancer), and may also be useful for understanding complex expression relationships between genes associated with a specific disease (e.g., cancer) and a mechanisms of action of drugs (e.g., therapeutic agents such as anticancer drugs) associated with a specific disease (e.g., cancer).

In addition, as the hierarchical relationship between genes is investigated, biomarkers that can be applied in actual clinical practice (e.g., previously unknown new biomarkers) may be easily discovered. In addition, a new relationship between genes may also be discovered. Furthermore, it is possible to more accurately predict therapeutic targets associated with a specific disease by building a more practical and accurate gene network than existing approaches.

Hereinafter, various embodiments of a method for analyzing a hierarchical relationship between two genes constituting a candidate gene pair will be described.

First, a method for analyzing a hierarchical relationship between genes according to some embodiments of the present disclosure will be described.

The present embodiments relate to a method for dividing two genes into an upstream gene and a downstream gene on the basis of the number of differentially expressed genes. For example, in the two genes, a first gene having a larger number of associated differentially expressed genes may be identified as an upstream gene, and a second gene having a smaller number of associated differentially expressed genes may be identified as a downstream gene. The reason is that a large number of differentially expressed genes associated with a certain gene means that the corresponding gene is highly likely to have the ability to regulate (control) the expression of other genes.

Hereinafter, the present embodiments will be described in more detail with reference to FIGS. 8 and 9.

FIGS. 8 and 9 are exemplary diagrams for describing analyzing a hierarchical relationship between two genes on the basis of the number of differentially expressed genes according to some embodiments of the present disclosure.

Referring to FIG. 8, the number of differentially expressed genes associated with each of a first gene (e.g., DTYMK) and a second gene (e.g., MRPL44) constituting a candidate gene pair may be calculated.

Specifically, first differentially expressed gene(s) may be identified (e.g., ‘100’ first differentially expressed genes are identified) by analyzing gene expression data between a first patient group with a high expression level of the first gene (i.e., high-expression patient group) and a second patient group with a low expression level of the first gene (i.e., low-expression patient group). The first patient group may consist of, for example, patients with a top X % expression level of the first gene (however, X is a real number greater than 0 and 50 or less), and the second patient group may consist of patients with a bottom X % expression level of the first gene, but the scope of the present disclosure is not limited thereto.

Next, second differentially expressed gene(s) may be identified (e.g., ‘100’ second differentially expressed genes are identified) by analyzing gene expression data between a third patient group with a high expression level of the second gene (i.e., high-expression patient group) and a fourth patient group with a low expression level of the second gene (i.e., low-expression patient group).

Next, third differentially expressed gene(s) may be identified (e.g., ‘100’ third differentially expressed genes are identified) by analyzing gene expression data between a fifth patient group with high expression levels of the first gene and the second gene (i.e., high-expression patient group) and a sixth patient group with low expression levels of the first gene and the second gene (i.e., low-expression patient group).

Next, a first common gene(s) may be identified between the first differentially expressed gene(s) and the third differentially expressed gene(s) (e.g., ‘79’ first common genes are identified). Here, the first common gene(s) may be understood as differentially expressed gene(s) associated with the first gene.

Next, a second common gene(s) may be identified between the second differentially expressed gene(s) and the third differentially expressed gene(s) (e.g., ‘31’ second common genes are identified). Here, the second common gene(s) may be understood as differentially expressed gene(s) associated with the second gene.

Next, the number of first common gene(s) and the number of second common gene(s) may be compared to identify upstream genes and downstream genes. For example, as illustrated in FIG. 8, when the number of first common gene(s) is greater than the number of second common gene(s), the first gene (DTYMK) may be identified as the upstream gene, and the second gene (MRPL44) may be identified as the downstream gene. That is, assuming that there is a set of genes that are affected from both the upstream genes and downstream genes, the number of genes affected from the upstream genes in the corresponding gene set has to be greater than the number of genes affected from the downstream genes (see hatched area). Therefore, it may be understood that genes with a greater number of common differentially expressed genes are identified as upstream genes.

As described above, in FIG. 8, it can be determined that the DTYMK gene is upstream of the MRPL44 gene (see arrow), and in FIG. 9, it can be determined that the DTYMK gene is upstream of the USMG5 gene (see arrow).

Meanwhile, in some embodiments, in the two genes constituting the candidate gene pair, the number of first candidate gene pairs to which the first gene belongs (i.e., it means the number of genes having a correlation with the first gene) may be calculated, and the number of second candidate gene pairs to which the second gene belongs (i.e., it means the number of genes having a correlation with the second gene) may be calculated. In addition, when the number of first candidate gene pairs is greater than or equal to the number of second candidate gene pairs, the first gene may be identified as the upstream gene and the second gene may also be identified as the downstream gene. When the first gene has a correlation with more genes than the second gene, the first gene is more likely to be an upstream gene than the second gene.

So far, the method for analyzing a hierarchical relationship between genes according to some embodiments of the present disclosure has been described with reference to FIGS. 8 and 9. As described above, it is possible to accurately analyze (investigate) the hierarchical relationship between two genes constituting a candidate gene pair on the basis of the number of differentially expressed genes.

Hereinafter, the method for analyzing a hierarchical relationship between genes according to some other embodiments of the present disclosure has been described with reference to FIG. 10 to 12.

The present embodiments relate to a method for dividing two genes into upstream and downstream genes by analyzing a temporal relationship between the expressions of the two genes according to drug administration (hereinafter, abbreviated as the ‘temporal relationship’ between the two genes). For example, in the two genes, the first gene whose expression changes first due to a specific drug may be identified as the upstream gene, and the second gene whose expression changes later may be identified as the downstream gene (because an increase in the expression level of the second gene as the expression level of the first gene increases is more likely to mean that the first gene regulates the expression of the second gene). Alternatively, in the two genes, the first gene that is expressed first by a specific drug may be identified as the upstream gene, and when a slope of the second gene that is expressed later (i.e., a slope representing a gene expression level over time) follows a slope pattern of the first gene, the second gene may be identified as the downstream gene.

Specifically, data on drug duration may be obtained to analyze a temporal relationship (difference) between two genes constituting a candidate gene pair. The data on drug duration may include, for example, data representing changes in gene expression levels according to drug administration (i.e., changes in gene expression levels over time), and such data may be obtained from a website such as clue.io. However, the scope of the present disclosure is not limited thereto. The drug may include, without limitation, any substance (e.g., an anticancer agent) that affects the expression of genes.

Next, the two genes may be divided into upstream and downstream genes by analyzing the drug duration data, which will be described with reference to FIG. 10.

FIG. 10 is an exemplary diagram for describing a method for analyzing a temporal relationship between two genes according to drug administration.

Referring to FIG. 10, it may be determined which gene has been expressed first, by analyzing a gene expression pattern that changes over time using drug duration data.

For example, assuming that the first gene is ‘gene A’ and the second gene is ‘gene B’, a first slope (i.e., a change a′ in expression level of the first gene over time in a first interval) of the first gene in the first interval (e.g., a time interval of up to 3 h) after administration of a specific drug is calculated, and a second slope (e.g., b′) of the second gene in the first interval may also be calculated. In addition, when the first slope is greater than or equal to the second slope, the first gene may be identified as the upstream gene and the second gene may be identified as the downstream gene.

As another example, assuming that the first gene is ‘gene A’ and the second gene is ‘gene B’, the first slope (e.g., a′) of the first gene in a first interval (e.g., time interval of DMSO-3 h) and a second slope (e.g., a″) of the first gene in a second interval (e.g., time interval of 3 h-6 h) after administration of a specific drug may be calculated. Next, a third slope (e.g., b′) of the second gene in the first interval and a fourth slope (e.g., b″) of the second gene in the second interval after administration of a specific drug may be calculated. Then, when the fourth slope (e.g., b″) follows the pattern of the first slope (e.g., a′), the first gene may be identified as the upstream gene and the second gene may be identified as the downstream gene. For example, when both the first slope and the fourth slope are 0.5 or greater and the third slope is 0.2 or less, the first gene and the second gene may be identified as the upstream gene and the downstream gene, respectively (i.e., it is determined that the fourth slope follows the pattern of the first slope), but the scope of the present disclosure is not limited thereto.

To further provide convenience of understanding, it will be described in detail by referring back to FIG. 10.

FIG. 10 illustrates a case where a drug is ‘Vorinostat’ and dimethyl sulfoxide (DMSO) is added together with the drug, and it is assumed that genes A and B are arbitrary genes constituting a candidate gene pair.

As illustrated in FIG. 10, on the basis of the gene expression levels upon addition of Vorinostat and dimethyl sulfoxide, the gene expression levels after 3 hours, and the gene expression levels after 6 hours, it can be inferred which gene in the candidate gene pair has been expressed first. Specifically, an average value of gene expression level (e.g., average of about 12,000 values) at each time point (when DMSO is added, after 3 h, or after 6 h) is calculated, and using these average values, the slopes (a′, b′) of the first interval (DMSO-3 h) and the slopes (a″, b″) of the second interval (3 h-6 h) may be calculated. In FIG. 10, a′ represents the slope in the first interval (DMSO-3 h) of gene A, a″ represents the slope in the second interval (3 h-6 h) of gene A, b′ represents the slope in the first interval (DMSO-3 h) of gene B, and b″ represents the slope in the second interval (3 h-6 h) of gene B. In addition, in FIG. 10, it is assumed that the slope in all intervals is greater than 0. In FIG. 10, dimethyl sulfoxide has been used as an additive, but the scope of the present disclosure is not limited thereto, and other additives may also be used.

When gene A is the upstream gene and gene B is the downstream gene, a pattern may appear in which gene A is expressed first (i.e., the change in expression level starts first), the slope a′ of gene A in the first interval (DMSO-3 h) is greater than the slope b′ of gene B, which is expressed later, and the slope a″ of gene A in the second interval (3 h-6 h) maintains a positive value. In addition, a phenomenon may occur in which the slope of gene B follows the slope pattern of gene A. For example, a phenomenon may occur in which the slope b″ in the second interval (3 h-6 h) of gene B follows the pattern of the slope a′ in the first interval (DMSO-3 h) of gene A. Therefore, using these temporal relationships, it is possible to accurately identify which gene is the upstream gene.

For example, when the slope a′ of gene A, which is expressed first, in the first interval (DMSO-3 h) is about 0.5 or more, the slope b′ of gene B, which is expressed later, is between 0 and about 0.2, and the slope b″ of gene B in the second interval (3 h-6 h) is about 0.5 or more, gene A and gene B may be identified as the upstream (or source) gene and the downstream (or target) gene, respectively. However, the scope of the present disclosure is not limited thereto. When analyzing the temporal relationship between two genes, different criteria may also be applied to the gene expressed first and the gene expressed later.

In addition, in drugs above a reference value (e.g., about 7) among 10 drugs, if gene A is identified as the upstream gene and gene B is identified as the downstream gene, gene A may be finally determined as the upstream gene and gene B may be finally determined as the downstream gene (see FIG. 11). As a result, the identification of the upstream and downstream genes may be more accurately performed.

Meanwhile, the above-described slope (Slope) may be defined as a change in gene expression level (GeneExpression) according to a change in time (Time (h)) as shown in the following Equation 1.

Slope=(ΔGeneExpression)/(ΔTime(h)) [Equation 1]

FIG. 11 illustrates results of analyzing a temporal relationship between an ECT2 gene and an MAD2L1 gene using 10 drugs.

Referring to FIG. 11, when drugs of Vorinostat, Afatinib, Erlotinib, Fulvestrant, HMN-214, Mitoxantrone, Lapatinib, Tanespimycin, Pazopanib, and Neratinib were administered together with dimethyl sulfoxide, the expression level of ECT2 generally increased first (see a DMSO-3 h interval), and thus the expression level of MAD2L1 increased subsequently (see a 3 h-6 h interval). Therefore, in FIG. 11, the ECT2 gene may be identified as the upstream (or source) gene, and the MAD2L1 gene may be identified as the downstream (or target) gene.

FIG. 12 illustrates a gene network built on the basis of the results of analyzing temporal relationships of all candidate gene pairs associated with lung cancer. In FIG. 12, it is assumed that a reference number of drugs to be finally determined as the upstream gene is ‘7’ (however, the total number of drugs is ‘10’). In addition, the size of each node constituting the gene network in FIG. 12 represents the number of associated downstream genes (i.e., the number of downstream genes influenced by the genes of the corresponding node) (i.e., the size of the node increases in proportion to the number), and the strength (e.g., thickness) of an edge represents the degree to which the upstream gene is associated with the downstream gene (e.g., the influence of the upstream gene on the downstream gene, which may be a correlation coefficient, etc.).

Referring to FIG. 12, the gene network may be expressed in a hierarchical structure, and the node of the top gene (e.g., ECT2) with the greatest influence may be disposed at the top of the gene network. Through the gene network, it may be determined at a glance how a specific gene (e.g., ECT2) affects the expression of other genes. For example, it may be determined at a glance that the ECT2 gene affects the expression of a total of six genes, including MAD2L1, HELLS, OPA1, ZWILCH, SPC25, and POLQ, and the degree of influence may also be determined at a glance.

So far, the method for analyzing a hierarchical relationship between genes according to some other embodiments of the present disclosure has been described with reference to FIG. 10 to 12. As described above, it is possible to accurately analyze (investigate) the hierarchical relationship between two genes constituting a candidate gene pair by analyzing a temporal relationship between the expressions of genes that are shown according to drug administration.

Hereinafter, a method for analyzing a hierarchical relationship between genes according to some other embodiments of the present disclosure has been described with reference to FIG. 13 to 15.

The present embodiments relate to a method for dividing two genes into an upstream gene and a downstream gene by analyzing a relationship between changes in expression levels of the two genes according to short hairpin RNA (shRNA) knockdown. For example, in the two genes, a first gene that is knocked down by a specific shRNA may be identified as the upstream gene, and a second gene that is downregulated regardless of the shRNA may be identified as the downstream gene.

Specifically, gene expression data (hereinafter, abbreviated as ‘shRNA data’) on shRNA knockdown may be obtained to analyze the relationship between changes in expression levels of two genes constituting a candidate gene pair (e.g., relationship between sequential changes in expression levels). Such gene expression data may include, for example, data representing changes in the expression levels of shRNA and genes resulting from shRNA, and such data may be obtained from a dataset such as GSE106127. However, the scope of the present disclosure is not limited thereto.

Next, shRNA data is analyzed to analyze the relationship between changes in the expression levels of the two genes, and as a result, the two genes may be divided into an upstream gene and a downstream gene.

For example, changes in expression levels of two genes due to knockdown shRNA may be identified by analyzing shRNA data tested in an A549 cell line. Here, a gene knocked down by shRNA may be identified as the upstream (or source) gene, and a gene downregulated together with the upstream gene (i.e., gene satisfying a predetermined condition) may be identified as the downstream (or target) gene. In other words, if there is a second gene that is downregulated when the first gene is knocked down by a specific shRNA, the first gene may be identified as the upstream gene and the second gene may be identified as the downstream gene. For example, when a z-score (i.e., a score on a gene expression level) of the second gene is negative (e.g., less than about −1), the second gene may be identified as a downstream gene of the first gene, but the scope of the present disclosure is not limited thereto.

As a more specific example, when the first gene of the two genes constituting the candidate gene pair is knocked down with a specific shRNA, the second gene as the other gene is downregulated, but when the second gene is knocked down with the specific shRNA, the first gene is not downregulated, and then the first gene may be identified as an upstream gene of the second gene.

To further provide convenience of understanding, it will be described in detail by referring back to FIGS. 13 and 14.

FIG. 13 schematically illustrates a method for identifying upstream genes and downstream genes on the basis of shRNA data, and FIG. 14 schematically illustrates identifying downstream genes on the basis of a z-score of genes according to shRNA. In FIG. 13, the upstream genes and the downstream genes are illustrated as ‘source genes’ and ‘target genes’, respectively.

Referring to FIGS. 13 and 14, it is possible to accurately infer which gene has been expressed first by analyzing a gene expression pattern that changes sequentially on the basis of shRNA data.

For example, when 3,724 genes knocked down by shRNA are set as upstream (or source) genes using shRNA data from an A549 cell line, and 978 landmark genes provided from an L1000 dataset of CMAP LINCS are set as downstream (or target) gene candidates, about 200,000 gene pairs may be constituted. In addition, among the 978 landmark genes, genes that meet a condition of having a z-score of less than about −1 may be considered as downstream (or target) genes. For reference, a negative z-score value means that the gene expression level decreased along with the upstream gene.

FIG. 15 illustrates a gene network built on the basis of the results of analyzing relationships between expression changes in candidate gene pairs using shRNA data. In FIG. 15, the size of each node constituting the gene network represents the number of downstream genes (i.e., the number of downstream genes influenced by the genes of the corresponding node) (i.e., the size of the node increases in proportion to the number), and the strength (e.g., thickness) of an edge represents the degree to which the upstream gene is associated with the downstream gene (e.g., the influence of the upstream gene on the downstream gene, which may be a z-score, etc.).

Referring to FIG. 15, it can be determined at a glance that a NUSAP1 gene is a gene that affects the expression of the most genes, followed by a CEP55 gene.

So far, the method for analyzing the hierarchical relationship between genes according to some other embodiments of the present disclosure has been described with reference to FIG. 13 to 15. As described above, it is possible to accurately analyze (investigate) the hierarchical relationship between two genes constituting a candidate gene pair by analyzing a relationship between changes in the expression levels of genes that are shown according to shRNA knockdown.

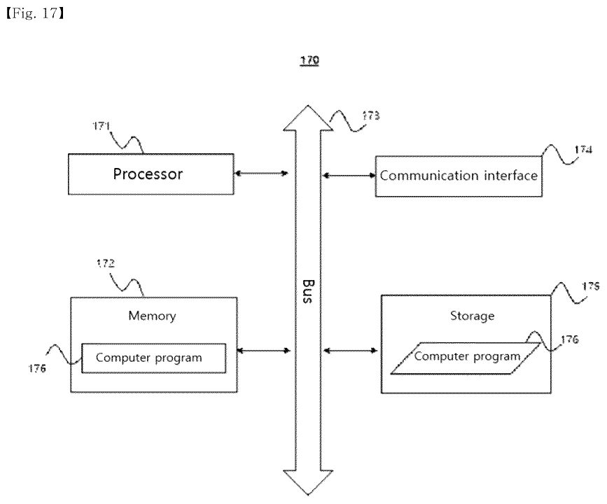

Hereinafter, Experimental Examples conducted by the present inventors will be described with reference to FIG. 16, etc.

### EXPERIMENTAL EXAMPLE

FIG. 16 schematically illustrates a genetic relationship analysis process performed in Experimental Examples. Hereinafter, it will be described with reference to FIG. 16.

First, for clinically applicable genetic screening, prognostic analysis was performed using data of 515 lung adenocarcinoma (LUAD) patients in TCGA. Prognostic analysis, correlation analysis, etc. were performed as described with reference to FIG. 2.

### 1) Prognostic Analysis

Using data of 515 lung adenocarcinoma patients in TCGA, as a result of performing prognostic analysis, 1,973 cancer-specific genes were identified.

Specifically, when a patient group with a high expression level of a specific gene (i.e., a high-expression patient group) had a poor prognosis, the specific gene was identified as a cancer-specific gene that served as a marker of the corresponding carcinoma. At this time, the gene expression level was divided from 5% to 50% at 5% intervals, and the survival rates between the high-expression patient group and the low-expression patient group were compared and analyzed (see the description in FIG. 3). In addition, when the survival rate of the high-expression patient group was significantly lower than that of the low-expression patient group, the corresponding gene was selected as a cancer-specific gene (i.e., an adverse gene).

Table 1 below shows some of results of screening cancer-specific genes by analyzing the prognosis of patients with lung adenocarcinoma. Referring to Table 1, an ABCC11 gene was selected as an adverse gene to survival (i.e., a cancer-specific gene) because it was confirmed that the survival rate of the high-expression patient group was significantly lower than that of the low-expression patient group in all 10 out of 10 analysis results. An ABAT gene was selected as a favor gene to survival because it was confirmed that the survival rate of the high-expression patient group was significantly higher than that of the low-expression patient group in all 10 out of 10 analysis results. It was confirmed that an A2BP1 gene had no significant difference in survival rate between the two patient groups in all 10 out of 10 analysis results.

TABLE 1

| Gene | Prognosis | Count | Selection |
|---|---|---|---|
| A2LD1 | [‘Not Sig’, ‘Adverse’, ‘Adverse’, ‘Adverse’, ‘Adverse’, ‘Adverse’, ‘Adverse’, ‘Not Sig’, ‘Not Sig’, ‘Not Sig’] | (0. 6, 4) | Adverse |
| A4GALT | [‘Not Sig’, ‘Not Sig’, ‘Adverse’, ‘Adverse’, ‘Adverse’, ‘Adverse’, ‘Adverse’, ‘Adverse’, ‘Adverse’, ‘Not Sig’] | (0, 7, 3) | Adverse |
| A2M | [‘Not Sig’, ‘Favor’, ‘Favor’, ‘Favor’, ‘Favor’, ‘Favor’, ‘Favor’, ‘Favor’, ‘Favor’, ‘Not Sig’] | (8, 0, 2) | Favor |
| ABAT | [‘Favor’, ‘Favor’, ‘Favor’, ‘Favor’, ‘Favor’, ‘Favor’, ‘Favor’, ‘Favor’, ‘Favor’, ‘Favor’] | (10, 0, 0) | Favor |
| ABCC11 | [‘Adverse’, ‘Adverse’, ‘Adverse’, ‘Adverse’, ‘Adverse’, ‘Adverse’, ‘Adverse’, ‘Adverse’, ‘Adverse’, ‘Adverse’] | (0, 10, 0) | Adverse |
| A2BP1 | [‘Not Sig’, ‘Not Sig’, ‘Not Sig’, ‘Not Sig’, ‘Not Sig’, ‘Not Sig’, ‘Not Sig’, ‘Not Sig’, ‘Not Sig’, ‘Not Sig’] | (0, 0, 10) | Not Sig |
| A2ML1 | [‘Not Sig’, ‘Not Sig’, ‘Not Sig’, ‘Not Sig’, ‘Not Sig’, ‘Not Sig’, ‘Adverse’, ‘Adverse’, ‘Not Sig’, ‘Not Sig’] | (0, 2, 8) | Not Sig |
| . | . | . | . |
| . | . | . | . |
| . | . | . | . |

### 2) Correlation Analysis

To analyze correlations between 1,973 cancer-specific genes, it was confirmed whether the expression levels of two genes had a linear relationship. In addition, among 1,973 cancer-specific genes, gene pairs with a correlation coefficient of approximately 0.6 or higher were selected as candidate gene pairs. Referring back to FIG. 7, it was confirmed that the ANXA2 and ANXA2P2 genes were a gene pair with the strongest correlation.

Table 2 below shows some of the results of analyzing the correlations between cancer-specific genes. In Table 2, the correlation refers to a correlation index.

TABLE 2

| Rank | Gene A | Gene B | Correlation |
|---|---|---|---|
| 1 | ANXA2P2 | ANXA2 | 0.97359052 |
| 2 | YEATS4 | CCT2 | 0.9667976 |
| 3 | MFSD2B | HMGA1 | 0.95059476 |
| 4 | COL5A2 | COL5A1 | 0.94849235 |
| 5 | EIF5AL1 | EIF5A | 0.94456471 |
| . | . | . | . |
| . | . | . | . |
| . | . | . | . |

### 3-1) Hierarchical Relationship Analysis-on Basis of Number of Differentially Expressed Genes

A hierarchical relationship between two genes constituting the identified candidate gene pairs was analyzed on the basis of the number of differentially expressed genes.

Specifically, the number of differentially expressed genes associated with the first gene and the number of differentially expressed genes associated with the second gene constituting the candidate gene pair were compared, and the gene with the larger number was identified as an upstream gene and the gene with the smaller number was identified as a downstream gene (see description in FIG. 8, etc.). Referring back to FIGS. 8 and 9, in an MRPL44 gene and a DTYMK gene, the DTYMK gene was identified as an upstream gene of the MRPL44 gene, and in a USMG5 gene and a DTYMK gene, the DTYMK gene was identified as an upstream gene of the USMG5 gene.

In addition, a gene network associated with lung adenocarcinoma was built by connecting nodes of upstream genes and nodes of downstream genes with directed edges.

### 3-2) Hierarchical Relationship Analysis—on Basis of Drug Administration

On the basis of a temporal relationship of genes (i.e., gene expression patterns that change over time) shown in drug duration data, a hierarchical relationship between two genes constituting candidate gene pairs was analyzed.

Specifically, a slope of genes in an L1000 dataset (i.e., changes in gene expression level over time) according to a drug duration was calculated for each drug. Among 19 cell lines provided by CMAP LINCS 2020, A549 was selected, and drug duration data for the A549 cell line were obtained from 10 drug duration databases. In addition, data on gene expression levels when 10 drugs and dimethyl sulfoxide were added to the A549 cell line, gene expression levels after 3 hours, and gene expression levels after 6 hours were analyzed, and the slopes were calculated for each interval. Specifically, an average value of the expression levels of approximately 12,000 genes at each time point (see DMSO, 3 h, and 6 h in FIG. 10) was obtained, and the slopes (see a′, b′, a″, and b″) of the DMSO-3 h interval and the 3 h-6 h interval were calculated using each average value. It was assumed that the slopes of all intervals were greater than 0.

In at least 7 out of 10 drugs, when the slope a′ of the DMSO-3 h interval of the gene A expressed first was 0.5 or higher, the slope b′ of the DMSO-3 h interval of the gene B expressed later was between 0 and 0.2, and the slope b″ of the gene B in the 3 h-6 h interval was 0.5 or higher, gene A and gene B were identified as the upstream gene and the target gene, respectively.

Table 3 shows upstream (or source) genes that are shown in the analysis results of 10 drugs, and Table 4 shows the number of downstream genes affected by the upstream genes.

TABLE 3

| Gene A | Gene B | [symbol] | [symbol] | [symbol] | [symbol] | [symbol] | [symbol] | [symbol] | [symbol] | [symbol] | [symbol] | Cnt(A) | Cnt(B) | [symbol] | Source |
|---|---|---|---|---|---|---|---|---|---|---|---|---|---|---|---|
| ECT2 | [symbol] | ECT2 | ECT2 | ECT2 | ECT2 | ECT2 | ECT2 | ECT2 | ECT2 | ECT2 | None | 9 | [symbol] | 9 | ECT2 |
| ECT2 | [symbol] | ECT2 | ECT2 | ECT2 | ECT2 | ECT2 | None | ECT2 | ECT2 | ECT2 | None | 8 | [symbol] | [symbol] | ECT2 |
| ECT2 | [symbol] | None | ECT2 | ECT2 | ECT2 | ECT2 | ECT2 | ECT2 | None | ECT2 | None | 7 | [symbol] | 7 | ECT2 |
| ECT2 | [symbol] | None | ECT2 | ECT2 | ECT2 | ECT2 | None | ECT2 | None | ECT2 | None | 6 | [symbol] | 6 | ECT2 |
| ECT2 | [symbol] | None | ECT2 | None | ECT2 | ECT2 | None | None | None | None | None | [symbol] | [symbol] | [symbol] | ECT2 |
| ECT2 | [symbol] | ECT2 | ECT2 | None | None | ECT2 | None | None | None | None | None | [symbol] | [symbol] | [symbol] | ECT2 |

| | | | |
|---|---|---|---|
| . | . | . | . |
| . | . | . | . |
| . | . | . | . |

[symbol]

indicates data missing or illegible when filed

TABLE 4

| Source | Full name | count |
|---|---|---|
| ECT2 | Epithelial Cell Transforming 2 | 6 |
| RACGAP1 | Rac GTPase Activating Protein 1 | 5 |
| RAD51AP1 | RAD51 Associated Protein 1 | 5 |
| TMPO | Thymopoietin | 5 |
| RRM2 | Ribonucleotide Reductase Regulatory Subun it M2 | 5 |
| MCM10 | Minichromosome Maintenance 10 Replication | 5 |
| | Initiation Factor | |
| CEP55 | Centrosomal Protein 55 | 5 |
| KIF4A | Kinesin Family Member 4A | 5 |
| ASPM | Assembly Factor For Spindle Microtubules | 5 |
| TTK | TTK Protein Kinase | 5 |
| HJURP | Holliday Junction Recognition Protein | 5 |
| NCAPG | Non-SMC Condensin I Complex Subunit G | 5 |
| ERCC6L | ERCC Excision Repair 6 Like, Spindle Assembly | 5 |
| | Checkpoint Helicase | |
| . | . | . |
| . | . | . |
| . | . | . |

In addition, a gene network associated with lung adenocarcinoma was built by connecting nodes of upstream genes and nodes of downstream genes with directed edges. It will be described with reference to FIG. 12.

3-3) Hierarchical Relationship Analysis—on Basis of shRNA

On the basis of a relationship between sequential expression level changes of genes shown in shRNA data, a hierarchical relationship between two genes constituting candidate gene pairs was analyzed.

Specifically, 3,724 genes knocked down by shRNA were set as upstream (or source) genes, and 978 landmark genes from the L1000 dataset provided by CMAP were set as candidates for downstream (or target) genes to constitute a total of 200,000 gene pairs. Then, using the shRNA database of the A549 cell line, when 3,724 genes were knocked down, genes that met a condition of having a z-score of less than −1 among 978 landmark genes were considered as downstream (or target) genes.

Table 5 below shows the number of downstream genes affected by the upstream genes, and Table 6 below shows pairs of upstream genes (see source) and downstream genes (see target) identified on the basis of shRNA data and z-scores (see Value).

TABLE 5

| Source | Full name | count |
|---|---|---|
| NUSAP1 | nucleolar and spindle associated protein 1 | 20 |
| CEPSS | centrosomal protein 55 kDa | 15 |
| TTK | TTK protein kinase | 13 |
| KIF20A | kinesin family member 20A | 8 |
| CDC25A | cell division cycle 25A | 8 |
| ERCC6L | ERCC Excision Repair 6 Like, Spindle | 6 |
| | Assembly Checkpoint Helicase | |
| PSRC1 | proline/serine-rich coiled-coil 1 | 6 |
| RAD51 | RAD51 homolog (RecA homolog, E. | 5 |
| | coli) (S. cerevisiae) | |
| BUB18 | BUB1 budding uninhibited by | 4 |
| | benzimidazoles 1 homolog beta (yeast) | |
| SNRPD1 | small nuclear ribonucleoprotein D1 | 4 |
| | polypeptide 16 kDa | |
| . | . | . |
| . | . | . |
| . | . | . |

TABLE 6

| Source | Target | Value | Source_prognosis | Target_prognosis | Correlation |
|---|---|---|---|---|---|
| HMMR | TOP2A | −6.82658 | Adverse | Adverse | 0.603511 |
| MCM8 | TOP2A | −3.02175 | Adverse | Adverse | 0.645205 |
| NUSAP1 | TIMELESS | −2.91987 | Adverse | Adverse | 0.7166553 |
| NUSAP1 | TOP2A | −2.80219 | Adverse | Adverse | 0.7963087 |
| CEP55 | NUSAP1 | −2.6824 | Adverse | Adverse | 0.6888808 |

| | | |
|---|---|---|
| . | . | . |
| . | . | . |
| . | . | . |

In addition, a gene network associated with lung adenocarcinoma was built by connecting nodes of upstream genes and nodes of downstream genes with directed edges. It will be described with reference to FIG. 15.

So far, Experimental Examples conducted by the present inventors have been described. Hereinafter, an exemplary computing device 170 capable of implementing the system 10 according to some embodiments of the present disclosure will be described with reference to FIG. 17.

FIG. 17 is an exemplary hardware configuration diagram illustrating the computing device 170.

As illustrated in FIG. 17, the computing device 170 may include one or more processors 171, a bus 173, a communication interface 174, a memory 172 that loads a computer program 176 executed by the processors 171, and a storage 175 that stores the computer program 176. However, only the components related to the embodiment of the present disclosure are illustrated in FIG. 17. Accordingly, those skilled in the art to which the present disclosure pertains can see that other general-purpose components other than components illustrated in FIG. 17 may be further included. That is, the computing device 170 may further include various components in addition to the components illustrated in FIG. 17. In addition, in some cases, the computing device 170 may also be configured in the form in which some of the components illustrated in FIG. 17 are omitted. Hereinafter, each component of the computing device 170 will be described.

The processor 171 may control the overall operation of each component of the computing device 170. The processor 171 may be configured to include at least one of a central processing unit (CPU), a micro processor unit (MPU), a micro controller unit (MCU), a graphic processing unit (GPU) or any type of processor well-known in the art. Further, the processor 171 may perform an operation of at least an application or program for executing the operation/method according to the embodiments of the present disclosure. The computing device 170 may provide one or more processors.

Next, the memory 172 may store various types of data, instructions, and/or information. The memory 172 may load the computer program 176 from the storage 175 in order to execute the operation/method according to the embodiments of the present disclosure. The memory 172 may be implemented as a volatile memory such as RAM, but the technical scope of the present disclosure is not limited thereto.

Next, the bus 173 may provide a communication function between components of the computing device 170. The bus 173 may be implemented as various types of buses, such as an address bus, a data bus, and a control bus.

Next, the communication interface 174 may support wired and wireless Internet communication of the computing device 170. In addition, the communication interface 174 may also support various communication schemes in addition to the Internet communication. To this end, the communication interface 174 may be configured to include a communication module well-known in the art.

Next, the storage 175 may non-temporarily store one or more computer programs 176. The storage 175 may be configured to include a nonvolatile memory such as a read only memory (ROM), an erasable programmable ROM (EPROM), an electrically erasable programmable ROM (EEPROM), a flash memory or the like, a hard disk, a removable disk, or any type of computer-readable recording medium well-known in the art to which the present disclosure pertains.

Next, the computer program 176 may include one or more instructions that allow the processor 171 to perform the operations/steps/methods according to various embodiments of the present disclosure when loaded on the memory 172. That is, the processor 171 may perform operations/steps/methods according to various embodiments of the present disclosure by executing one or more of the loaded instructions.

For example, the computer program 176 may include instructions to perform the operations of obtaining gene expression data and prognostic data of patients associated with a specific cancer; identifying cancer-specific genes associated with the specific cancer on the basis of the gene expression data and the prognostic data; analyzing correlations between the cancer-specific genes to identify candidate gene pairs in which positive or inverse correlations between the cancer-specific genes are established; analyzing a hierarchical relationship between the two genes constituting each of the candidate gene pairs; and building a gene network associated with the specific cancer on the basis of the results of the analysis.

As another example, the computer program 176 may include instructions to perform at least some of the operations/steps/methods described with reference to FIG. 1 to 16.

As exemplified, the system 10 according to some embodiments of the present disclosure may be implemented through the computing device 170.

So far, the exemplary computing device 170 capable of implementing the system 10 according to some embodiments of the present disclosure has been described with reference to FIG. 17.

So far, the technical ideas of the present disclosure described with reference to FIG. 1 to 17 may be implemented as computer-readable codes on non-transitory computer-readable recording media. The non-transitory computer-readable recording media may be, for example, removable recording media (CD, DVD, Blu-ray disc, USB storage device, removable hard disk) or fixed recording media (ROM, RAM, computer-mounted hard disk). The computer programs recorded on the non-transitory computer-readable recording media may be transmitted to another computing device through a network such as the Internet and installed on the computing device, and thus may be used on the computing device.

Hereinabove, even though it has been described that all of components constituting the embodiment of the present disclosure are coupled as a single unit or coupled to be operated as the single unit, the technical idea of the present disclosure is not necessarily limited to these embodiments. That is, one or more of all of the components may be selectively coupled to be operated within the scope of the object of the present disclosure.

Although operations are illustrated in the drawings in a particular order, it should not be understood to mean that the operations need to be performed in the illustrated particular order or in a sequential order, or that all of the illustrated operations need to be performed to obtain desired results. In a certain situation, multitasking and parallel processing may be advantageous. Moreover, in the embodiments described above, it should not be understood that the separation of the various components is not necessarily required, and it should be understood that the described program components and systems may generally be integrated together into a single software product or packaged into a plurality of software products.

All documents including publications, patent applications, patents, etc. cited in the present disclosure are illustrated by combining each cited reference individually and specifically, or combined with the present disclosure in the same manner as those combined and indicated in the present disclosure as a whole.

In order to understand the present disclosure, reference numerals are given in the preferred embodiments shown in the drawings, specific terms have been used to describe the embodiments of the present disclosure, but the present disclosure is not limited by the specific terms, and the present disclosure may include all components commonly conceived by those skilled in the art.

The present disclosure may be represented by functional block configurations and various processing steps. These functional blocks may be implemented as various numbers of hardware or/and software configurations for executing specific functions. For example, the present disclosure may adopt IC configurations, such as a memory, a processing, a logic, a look-up table, and the like, which may execute various functions by control of one or more microprocessors or other control devices.

The components of the present disclosure include various algorithms implemented in combination of a data structure, processes, routines, or other programming configurations, like being executed by software programming or software elements to be implemented by a programming or scripting language such as C, C++, Java, assembler, and the like. Functional aspects may be implemented as algorithms executed in one or more processors. In addition, the present disclosure may adopt the related art for electronic environment configuration, signal processing, and/or data processing. The terms “mechanism”, “element”, “means”, and “configuration” can be widely used and are not limited to mechanical and physical configurations. The terms may include the meaning of a series of processes (routines) of software in conjunction with a processor or the like.

The specific implementations described in the present disclosure are exemplary embodiments, and do not limit the scope of the present disclosure in any way. For brevity of the specification, descriptions of conventional electronic configurations, control systems, software, and other functional aspects of the systems may be omitted. In addition, the connection or connection members of lines between the components illustrated in the drawings exemplarily represent functional connections and/or physical or circuit connections, and may be illustrated as various functional connections, physical connections, or circuit connections that can be replaced or added in an actual device. In addition, unless specifically stated, such as “essential” or “important”, components may not necessarily be required for the application of the present disclosure.

As used in the present specification of the present disclosure (especially, in the appended claims), the use of the term “the” and similar instruction terms thereto may correspond to both singular and plural references. Also, in the present disclosure, when the range is described, the invention applied with individual values belonging to the above range is included (unless expressly indicated otherwise) and therefore, each individual value configuring the range will be disclosed in the detailed description of the invention. Finally, unless the order is explicitly stated or stated to the contrary for steps configuring the method according to the present disclosure, the steps may be performed in any suitable order. The present disclosure is not necessarily limited to the order of description of the steps. All examples described herein or the terms indicative thereof (“for example”, etc.) used herein are merely to describe the present disclosure in more detail. Therefore, the scope of the present disclosure is not limited to the examples or exemplary terms unless limited by the appended claims. Also, it should be apparent to those skilled in the art that various modifications, combinations, and alternations can be made depending on design conditions and factors within the scope of the appended claims or equivalents thereof.

The apparatus described above may be implemented in hardware components, software components, and/or a combination of hardware components and software components. For example, the device and the components described in the embodiments may be implemented using, for example, one or more general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or other any devices capable of executing and responding instructions. A processing device may execute an operating system (OS) and one or more software applications running on the operating system. In addition, the processing device may also access, store, manipulate, process, and generate data in response to execution of software. For convenience of understanding, one processing device may be described to be used, but it can be seen to those skilled in the art that the processing device may include a plurality of processing elements and/or a plurality of types of processing elements. For example, the processing device may include a plurality of processors or one processor or one controller. In addition, other processing configurations, such as a parallel processor, are also possible.

Hereinabove, the embodiments of the present disclosure have been described with the accompanying drawings, but it can be understood by those skilled in the art that the present disclosure can be executed in other detailed forms without changing the technical idea or requisite features of the present disclosure. Therefore, it should be appreciated that the aforementioned embodiments described above are illustrative in all aspects and are not restricted. The protection scope of the present disclosure should be interpreted by the appended claims and all technical ideas in the equivalent range thereto should be interpreted to be included in the claims of the technical idea defined by the present disclosure.

1. A method for analyzing genetic relationships, the method performed by at least one computing device, comprising:
obtaining gene expression data and prognostic data of patients associated with a specific disease or condition;
identifying disease or condition-specific genes associated with the specific disease or condition on the basis of the gene expression data and the prognostic data;
analyzing correlations between the disease or condition-specific genes to identify candidate gene pairs in which positive or inverse correlations between the disease or condition-specific genes are established;
analyzing a hierarchical relationship between the two genes constituting each of the candidate gene pairs; and
building a gene network associated with the specific disease or condition on the basis of the results of the analysis.

2. The method for analyzing genetic relationships of claim 1, wherein the identifying of the disease or condition-specific genes includes:
analyzing prognosis of a first patient group and a second patient group on the basis of the gene expression data and the prognostic data, in which the first patient group is a patient group in which an expression level of a specific gene is higher than that of the second patient group; and
identifying the specific gene as a disease or condition-specific gene on the basis of the analysis results of the prognosis.

3. The method for analyzing genetic relationships of claim 2, wherein the analyzing of the prognosis includes:
analyzing survival rates between the first patient group having the expression level of the specific gene of top X % (however, X is a real number greater than 0 and 50 or less) and the second patient group having the expression level of the specific gene of bottom X %.

4. The method for analyzing genetic relationships of claim 1, wherein the identifying of the candidate gene pairs includes:
identifying the two genes having the positive correlation among the disease or condition-specific genes as the candidate gene pair.

5. The method for analyzing genetic relationships of claim 1, wherein the analyzing of the hierarchical relationship between the two genes includes:
identifying a first gene as an upstream gene and identifying a second gene as a downstream gene when the number of candidate gene pairs to which the first gene belongs is greater than or equal to the number of candidate gene pairs to which the second gene belongs.

6. The method for analyzing genetic relationships of claim 1, wherein the analyzing of the hierarchical relationship between the two genes includes:
dividing the two genes into an upstream gene and a downstream gene on the basis of the number of differentially expressed genes (DEGs) associated with each of the two genes.

7. The method for analyzing genetic relationships of claim 6, wherein the dividing of the two genes into the upstream gene and the downstream gene includes:
identifying a first differentially expressed gene between a first patient group and a second patient group, which is divided by an expression level of a first gene;
identifying a second differentially expressed gene between a third patient group and a fourth patient group, which is divided by an expression level of a second gene;
identifying a third differentially expressed gene between a fifth patient group and a sixth patient group, which is divided by the expression levels of the first gene and the second gene, in which the fifth patient group is a patient group in which the expression levels of both the first gene and the second gene are higher than those of the sixth patient group; and
identifying the upstream gene and the downstream gene on the basis of the first differentially expressed gene, the second differentially expressed gene, and the third differentially expressed gene.

8. The method for analyzing genetic relationships of claim 7, wherein the identifying of the upstream gene and the downstream gene includes:
deriving the number of first common genes between the first differentially expressed gene and the third differentially expressed gene and the number of second common genes between the second differentially expressed gene and the third differentially expressed gene; and
identifying the first gene as the upstream gene and identifying the second gene as the downstream gene, when the number of the first common genes is greater than or equal to the number of the second common genes.

9. The method for analyzing genetic relationships of claim 1, wherein the analyzing of the hierarchical relationship between the two genes includes:
dividing the two genes into an upstream gene and a downstream gene by analyzing a temporal relationship between expressions of the two genes according to a specific drug on the basis of drug duration data.

10. The method for analyzing genetic relationships of claim 9, wherein the dividing of the two genes into the upstream gene and the downstream gene includes:
calculating a first slope of a first gene at a first interval after administration of the specific drug, in which the first slope represents a change in an expression level of the first gene over time at the first interval;
calculating a second slope of a second gene at the first interval after the administration of the specific drug, in which the second slope represents a change in an expression level of the second gene over time at the first interval; and
identifying the first gene as the upstream gene and identifying the second gene as the downstream gene, when the first slope is greater than or equal to the second slope.

11. The method for analyzing genetic relationships of claim 10, wherein the dividing of the two genes into the upstream gene and the downstream gene includes:
calculating the first slope of the first gene at the first interval and the second slope of the first gene at a second interval after the administration of the specific drug, in which the first slope and the second slope represent changes in the expression level of the first gene over time at the first interval and the second interval, respectively, and the second interval is an interval subsequent to the first interval;
calculating a third slope of the second gene at the first interval and a fourth slope of the second gene at the second interval after the administration of the specific drug, in which the third slope and the fourth slope represent changes in the expression level of the second gene over time at the first interval and the second interval, respectively; and
identifying the first gene as the upstream gene and identifying the second gene as the downstream gene, when the fourth slope follows a pattern of the first slope.

12. The method for analyzing genetic relationships of claim 11, wherein both the first slope and the fourth slope are 0.5 or greater, and
the third slope is less than or equal to 0.2.

13. The method for analyzing genetic relationships of claim 1, wherein the analyzing of the hierarchical relationship between the two genes includes:
dividing the two genes into an upstream gene and a downstream gene by analyzing a relationship between changes in expression levels of the two genes according to a specific shRNA on the basis of the gene expression data on short hairpin RNA (shRNA) knockdown.

14. The method for analyzing genetic relationships of claim 13, wherein the dividing of the two genes into the upstream gene and the downstream gene includes:
identifying a first gene knocked down by the specific shRNA as the upstream gene, and identifying a second gene as the downstream gene when the second gene satisfies a predetermined condition.

15. The method for analyzing genetic relationships of claim 14, wherein the predetermined condition includes a condition in which a z-score of the second gene is negative when the first gene has been knocked down.

16. The method for analyzing genetic relationships of claim 14, wherein the predetermined condition includes a condition in which a z-score of the second gene is less than −1 when the first gene has been knocked down.

17. The method for analyzing genetic relationships of claim 14, wherein the predetermined condition includes a condition in which expression of the second gene is downregulated when the first gene has been knocked down, but the first gene is not downregulated when the second gene has been knocked down by another shRNA.

18. The method for analyzing genetic relationships of claim 1, wherein the building of the gene network associated with the specific disease or conditions includes:
generating a first node and a second node corresponding to a first gene identified as an upstream gene and a second gene identified as a downstream gene in the two genes as a result of the analysis, respectively; and
building the gene network by connecting the first node and the second node with a directed edge, in which the directed edge is an edge from the first node to the second node.

19. The method for analyzing genetic relationships of claim 18, further comprising:
selecting a node having an out-degree greater than or equal to a threshold among a plurality of nodes constituting the gene network; and
determining a gene corresponding to the selected node as a target gene for treating the specific disease or condition.

20. The method for analyzing genetic relationships of claim 18, further comprising:
selecting a node having an out-degree below a threshold among a plurality of nodes constituting the gene network; and
determining a gene corresponding to the selected node as a target gene for treating the specific disease or condition.

21. A system for analyzing genetic relationships comprising:
one or more processors; and
a memory storing a computer program executed by the one or more processors,
wherein the computer programs include instructions for operations of:
obtaining gene expression data and prognostic data of patients associated with a specific disease or condition;
identifying disease or condition-specific genes associated with the specific disease or condition on the basis of the gene expression data and the prognostic data;
analyzing correlations between the disease or condition-specific genes to identify candidate gene pairs in which positive or inverse correlations between the disease or condition-specific genes are established;
analyzing a hierarchical relationship between the two genes constituting each of the candidate gene pairs; and
building a gene network associated with the specific disease or condition on the basis of the results of the analysis.

22. A non-transitory computer-readable recording medium recorded with a computer program for genetic relationship analysis,
wherein the computer programs include instructions for operations of:
obtaining gene expression data and prognostic data of patients associated with a specific disease or condition;
identifying disease or condition-specific genes associated with the specific disease or condition on the basis of the gene expression data and the prognostic data;
analyzing correlations between the disease or condition-specific genes to identify candidate gene pairs in which positive or inverse correlations between the disease or condition-specific genes are established;
analyzing a hierarchical relationship between the two genes constituting each of the candidate gene pairs; and
building a gene network associated with the specific disease or condition on the basis of the results of the analysis.
